(19) United States
(12) Patent Application Publication
(10) Pub. No.: US 2023/0264240 A1
(43) Pub. Date: Aug. 24, 2023

(54) CLEANING BASKET FOR CLEANING BREATHING APPARATUS

(71) Applicant: MEIKO MASCHINENBAU GMBH & CO. KG, Offenburg (DE)

(72) Inventor: Bernd Ringwald, Ortenberg (DE)

(73) Assignee: MEIKO Maschinenbau GmbH and Co. KG, Offenburg (DE)

(21) Appl. No.: 18/004,244

(22) PCT Filed: Jul. 7, 2021
(86) PCT No.: PCT/EP2021/068790

§ 371 Date: Jan. 4, 2023

(30) Foreign Application Priority Data
Jul. 8, 2020 (DE) 10 2020 208 546.3

(51) Int. Cl. B08B 13/00; B08B 3/02; G01D 5/14; A61L 2/26; A61L 2/18

(52) U.S. Cl. CPC B08B 13/00; B08B 3/02; G01D 5/14; A61L 2/26; A61L 2/18; A61L 2202/17; A61L 2202/14; A61L 2202/123

(57) ABSTRACT

A cleaning basket (114), a cleaning device (116), a cleaning system (110) and a method for cleaning breathing apparatus (112) are proposed. The cleaning basket (114) comprises at least one basket rack (118), and the cleaning basket (114) further comprises at least one pressure attachment (120) connectable to at least one gas-conveying element (113) of the breathing apparatus (112), wherein the gas-conveying element (113) can have pressurized gas applied to it at least while the breathing apparatus (112) is subjected to at least one cleaning fluid (122), wherein the cleaning basket (114) has at least one pressure sensor (124), wherein the pressure sensor (124) is configured to detect when pressure is applied to the pressure attachment (120), wherein the cleaning basket (114) furthermore has at least one transmission device (126) for transmitting at least one result of the pressure application detection to a cleaning device (116).

### TECHNICAL FIELD

The invention relates to a cleaning basket, a cleaning device and a cleaning system for cleaning a breathing apparatus or component parts of a breathing apparatus. Furthermore, the invention relates to a method for cleaning a breathing apparatus. Such devices and methods can be generally used for cleaning a breathing apparatus or component parts of same, for example a breathing apparatus for emergency services, such as firefighters, technical aid agencies or paramedics, breathing apparatuses for divers or generally for people who work in hostile or critical environments and for members of military forces and security forces, such as, for example, the police. The proposed devices and methods can also be used for breathing apparatuses in the medical sector, for example breathing masks for supplying oxygen and for carrying out operations. Other fields of use are also conceivable.

### TECHNICAL BACKGROUND

Breathing apparatuses, such as, for example, protective breathing masks or breathing regulators, generally form part of the personal protective equipment of, for example, emergency services, military forces or security forces. For instance, a multiplicity of breathing apparatuses for various intended uses are known from the prior art. For example, emergency services, such as firefighters, use protective breathing masks with filters for removing harmful constituents from the respiratory air that is taken in. In many cases, however, what is referred to as a breathing regulator is used as an alternative or in addition to a filter, allowing the user to be ventilated with a respiratory gas, for example pressurized air. Breathing regulators, which are frequently also referred to as respiratory regulators, generally enable a user to breathe from a compressed gas cylinder or some other compressed gas connection and in this way to stay underwater, for example, or in some other atmosphere that cannot be breathed in or is toxic. For this purpose, the compressed gas, for example compressed air, from the compressed gas connection is adapted by the breathing regulator to a pressure prevailing in the user's working environment.

After each time they are used, the breathing apparatuses or component parts of same must generally be cleaned, sanitized, dried, checked, and repaired if necessary, and packed. The cleaning is intended to remove all contamination as a result of use or storage, so that the breathing apparatuses can be provided in a macroscopically clean and hygienically satisfactory state, for example, for the next steps of preparation. These requirements generally also apply to other component parts of breathing apparatuses, such as, for example, add-on and accessory parts of breathing masks, such as, for example, filters or breathing regulators.

In many cases, breathing apparatuses, such as, for example, breathing masks and accessories thereof, are either cleaned manually or washed in modified laundry washing machines with the aid of protective bags and/or with the aid of adaptors. For example, EP 0 935 687 B1 generally discloses a washing machine which has a tub with a drum. A shell of the drum has a bulged structure directed toward the interior of the drum, with holes arranged at corner points of outer contours of the bulging directed toward the exterior of the drum. With such washing machines, it is possible in principle to clean items of equipment for emergency services particularly gently.

EP 1 088 928 A1 discloses a holding system for protective breathing masks in a laundry treatment machine. The holding system has a carrying strut, which is arranged in a drum of the laundry treatment machine so as to rotate therewith, and to which the protective breathing masks can be connected.

DE 200 03 743 U1 and DE 298 22 172 U1 respectively disclose devices for treating protective suits. In these cases, clothes hangers which comprise flexible air outlet nozzles are used. The clothes hangers are respectively fastened to a pivoting device. Cleaning of breathing apparatuses is generally not possible, or only with difficulty, by means of the devices shown.

DE 10 2005 033 618 B3 discloses a device for cleaning protective breathing masks. The device comprises a closable housing and at least one receptacle, which is arranged in a carrier, for at least one protective breathing mask. A nozzle arrangement and a brush arrangement are also provided, with a movement of the protective breathing masks allowing them to be brushed. However, an individual assignment and cleaning of accessory parts of masks is not possible by means of the disclosed device. Furthermore, the cleaning of gas-conducting elements, such as, for example, breathing regulators, is not possible with the disclosed device. DE 200 03 744 U1 also discloses a device for cleaning, disinfecting and drying protective breathing masks that has a carrying frame with an assigned nozzle system and individual treatment locations. This device is also not suitable in principle for cleaning gas-conducting elements and accessory parts.

DE 10 2007 009 936 A1 discloses a cleaning device for compressed air breathing apparatuses. This device has a receiving space bounded by a protective grid and rotating nozzle carriers. The nozzle carriers are in this case located outside the protective grid. However, a disadvantage of the device presented is that cleaning liquid can penetrate into gas-conducting regions.

By contrast, the cleaning of sensitive components of breathing apparatuses, such as, for example, breathing regulators, is generally performed manually. If necessary, manual cleaning can be assisted by placement in ultrasonic cleaning devices. However, DE 10 2007 012 768 B4 discloses a method and a device for cleaning breathing regulators. In this case, the items to be cleaned are fitted onto holders of a rotating element and repeatedly immersed in a liquid bath with cleaning liquid, disinfecting liquid and rinsing liquid. Breathing regulators are first of all charged here with compressed air to provide a seal between valve and tube connection and are then immersed in the liquid bath. However, a disadvantage of such immersion methods is that complex holders with corresponding actuators are required in order to ensure by appropriate movements that cleaning fluid is removed from the various cavities after cleaning.

DE 100 20 835 A1 describes a device for treating protective breathing masks. A receptacle for populating with protective breathing masks is provided, in which the protecting breathing masks are coupled to the receptacle by means of the breathing apparatus connection. It is furthermore described that a collecting device for collecting treatment agents, and a pump for supplying the treatment agents, are provided in a compartment of the device.

U.S. Pat. No. 3,881,503 A discloses a device for washing and decontaminating items of anesthetic equipment. A nozzle system is provided by means of which water jets can be sprayed at high pressure onto the items for cleaning.

DE 11 74 169 B describes a device for cleaning protective breathing masks. In this case, a housing is provided with a rotatably mounted tubular frame, in which the masks for cleaning are stretched over holders. By means of the rotary frame, the protective breathing masks are moved with a rotational movement through a bath of a cleaning liquid in a tub.

WO 2011/144518 A2 describes a cleaning device for cleaning breathing apparatuses. The cleaning device comprises at least one cleaning chamber for receiving at least one breathing apparatus, and also at least one fluid device for applying at least one cleaning fluid to the breathing apparatus. The cleaning device furthermore has at least one pressurization device with at least one pressure connection. The pressure connection is connectable to at least one gas-conducting element of the breathing apparatus. The pressurization device is configured to charge the gas-conducting element with compressed gas.

DE 10 2015 108 857 A1 describes a method for treating at least one protective suit, in particular a chemical protective suit. The method comprises the following steps: a) at least one treatment adaptor is connected in a releasable and sealing manner to at least one opening of the protective suit, b) a cleaning and disinfecting fluid is conducted via the at least one treatment adapter into the interior of the protective suit, and the cleaning and disinfecting fluid conducted into the interior of the protective suit is discharged from the protective suit, c) a drying gas for drying the protective suit is conducted into the interior of the protective suit via the at least one treatment adaptor, and the drying gas which is conducted into the interior of the protective suit is discharged from the protective suit, d) the at least one treatment adaptor is separated from the at least one opening of the protective suit.

Despite the advantages which have been and are achieved with the above-described devices and methods, numerous technical challenges remain. There is the risk of a malfunction and of technical defects even when providing a seal or pressurizing gas-conducting elements during the cleaning operation. This may lead to cleaning fluid being able to enter gas-conducting component parts of the breathing apparatus. This conceals the risk that subsequently, during use, the cleaning fluid may freeze due to expanding compressed gases, for example compressed air, as a result of which the supply of respiratory air necessary for living may be interrupted.

### OBJECT OF THE INVENTION

It would therefore be desirable to provide devices and methods for cleaning a breathing apparatus that at least substantially avoid the disadvantages of known devices and methods of the type mentioned. In particular, the intention is to ensure, with as little apparatus outlay as possible and with a high degree of reliability, that cleaning fluid cannot penetrate gas-conducting elements of the breathing apparatus during the cleaning operation.

### GENERAL DESCRIPTION OF THE INVENTION

This object is addressed by a cleaning basket, a cleaning device, a cleaning system and a method for cleaning a breathing apparatus or else component parts of a breathing apparatus, having the features of the independent patent claims. Advantageous developments, which can be realized individually or in any desired combination, are set out in the dependent claims.

Hereinafter, the terms “have”, “comprise”, “encompass” or “include” or any grammatical deviations therefrom are used in a non-exclusive way. Accordingly, these terms can refer either to situations in which, besides the features introduced by these terms, no further features are present, or to situations in which one or more further features are present. By way of example, the expression “A has B”, “A comprises B”, “A encompasses B” or “A includes B” can refer either to the situation in which, apart from B, no further element is present in A (i.e. to a situation in which A exclusively consists of B), or to the situation in which, in addition to B, one or more further elements are present in A, for example element C, elements C and D or even further elements.

Furthermore, it is pointed out that the terms “at least one” and “one or more” and grammatical modifications of these terms, if they are used in association with one or more elements or features and are intended to express the fact that the element or feature can be provided singly or multiply, are generally used just once, for example when the feature or element is introduced for the first time. When the feature or element is subsequently mentioned again, the corresponding term “at least one” or “one or more” is generally no longer used, without this restricting the possibility that the feature or element can be provided singly or multiply.

Furthermore, hereinafter, the terms “preferably”, “in particular”, “for example” or similar terms are used in connection with optional features, without alternative embodiments thereby being restricted. In this regard, features introduced by these terms are optional features and there is no intention for the scope of protection of the claims, and in particular of the independent claims, to be restricted by these features. In this regard, the invention, as will be recognized by a person skilled in the art, can also be carried out using other configurations. In a similar manner, features introduced by “in one embodiment of the invention” or by “in one exemplary embodiment of the invention” are understood as optional features, without alternative configurations or the scope of protection of the independent claims being intended to be restricted thereby. Furthermore, these introductory expressions are intended to have no effect on all possibilities of combining the features introduced thereby with other features, whether they be optional or non-optional features.

In a first aspect, a cleaning basket for cleaning a breathing apparatus is proposed. The cleaning basket comprises at least one basket rack. Furthermore, the cleaning basket comprises at least one pressure connection which is connectable to at least one gas-conducting element of the breathing apparatus. The gas-conducting element can be charged with compressed gas at least while at least one cleaning fluid is being applied to the breathing apparatus. Furthermore, the cleaning basket has at least one pressure sensor. The pressure sensor is configured to identify a pressurization of the pressure connection. The cleaning basket furthermore has at least one transmission device for transmitting at least one result of the identification of the pressurization to a cleaning device.

The term “cleaning basket”, as is used here, also referred to as “flushing basket”, is a broad term which is to be given its customary and common meaning, as understood by a person skilled in the art. The term is not restricted to a specific or adapted meaning. The term may, without restriction, refer in particular to a portable receiving device, in which items for cleaning can be accommodated, can be introduced into a cleaning device, for example a dishwasher, and can subsequently be removed again from the cleaning device in a cleaned state. The cleaning basket can therefore be introduced reversibly into the cleaning chamber. Accordingly, the cleaning chamber can have, for example, at least one receptacle for the cleaning basket. Alternatively, the cleaning basket may also be installed, for example, permanently in a cleaning device. For example, the cleaning basket can be fastened in a rail system of the cleaning device and can be pushed via said rail system into a cleaning chamber of the cleaning device and pulled out again from the cleaning chamber following cleaning. Furthermore, cleaning baskets can also be used for temporary storage of items for cleaning. The cleaning basket can in particular have a multiplicity of openings such that liquid can drip downward. The cleaning basket can have in particular a substantially cuboidal configuration, with a basket base which, for example, can have a square or generally rectangular outline, and which has a supporting surface for placing the cleaning basket onto a flat work surface. Optionally, one or more basket walls can extend upward from the basket base, for example with a protruding edge and furthermore optionally other elements, such as, for example, handles. Furthermore, the cleaning basket can have a plurality of mask holder or other holders for items for cleaning, in particular the breathing apparatus. In addition, the cleaning basket, as described above and as will be explained in more detail below, has the at least one pressure connection.

The term “cleaning”, as is used here, is a broad term which is to be given its customary and common meaning, as understood by a person skilled in the art. The term is not restricted to a specific or adapted meaning. The term may, without restriction, refer in particular to an operation in which items for cleaning are freed from adhering macroscopic or even microscopic impurities and/or in which such impurities are at least partially eliminated. In addition, a disinfecting effect can optionally be carried out.

The term “breathing apparatus”, as is used here, is a broad term which is to be given its customary and common meaning, as understood by a person skilled in the art. The term is not restricted to a specific or adapted meaning. The term may, without restriction, refer in particular to a device which is configured in a suitable manner for protecting the respiratory tracts of a user from harmful influences and/or for charging the respiratory tracts of the user with one or more respiratory gases. For example, the breathing apparatus may be a device which is configured for the complete or partial filtering of ambient air and/or for the provision of respiratory gas to at least one user. These breathing apparatuses may be complete, operationally ready breathing apparatuses, or else component parts thereof, and consequently no distinction is made below, in terms of terminology, between breathing apparatuses and their component parts.

The breathing apparatus may in particular encompass at least one protective breathing mask, also referred to as a breathing mask. The term “protective breathing mask”, as is used here, is a broad term which is to be given its customary and common meaning, as understood by a person skilled in the art. The term is not restricted to a specific or adapted meaning. The term may, without restriction, refer in particular to a device which is a component part of a breathing apparatus and which is configured to cover all or part of a user's face, in particular a mouth portion and/or nose portion. The covering can serve in particular for protecting the respiratory tracts of the user against harmful substances and/or for conducting respiratory gases into the respiratory tracts and/or conducting respiratory gases out of the respiratory tracts. The protective breathing mask can furthermore cover further portions of the face and/or head of the user, for example the eyes. Accordingly, the protective breathing mask may in particular also have at least one transparent viewing window. The protective breathing mask can furthermore have at least one seal, for sealing an interior space of the protective breathing mask from an exterior, wherein the interior space is bounded, for example during operation of the protective breathing mask, by the protective breathing mask itself, the user's head and the seal of the protective breathing mask. In particular, the protective breathing mask can be of convex design, with at least one cavity being formed which is partially closed off by the protective breathing mask and the edges of which are formed, for example, by the at least one seal. In particular, the protective breathing mask can have a wall which is preferably configured completely or partially flexibly and which covers the mouth and nose and optionally further regions of the face, for example the eyes. The seal can have in particular at least one sealing lip which seals the protective breathing mask against the face such that said interior space between face and protective breathing mask is designed as a sealed region. Furthermore, the protective breathing mask, as will be explained in more detail below, can have at least one fixing for fastening on the head of the user, for example at least one belt, at least one fixing band or at least one fastening which consists of a plurality of bands and is frequently referred to as banding or webbing. The breathing mask can have, for example, at least one opening and can be configured, for example, to enable an air supply or respiratory gas supply. For example, the respiratory mask can have at least one thread for the connection of a respiratory gas supply. Furthermore, the protective breathing mask can have, for example, at least one exhalation valve. The protective breathing mask can be produced, for example, entirely or partially from a flexible material, in particular from at least one elastomer material, for example rubber and/or silicone.

Furthermore, the breathing apparatus can have at least one gas-conducting element. The term “gas-conducting element”, as is used here, is a broad term which is to be given its customary and common meaning, as understood by a person skilled in the art. The term is not restricted to a specific or adapted meaning. The term may, without restriction, refer in particular to an element or a combination of elements which are charged with a respiratory gas during use of the breathing apparatus by a human or an animal user or can come into contact with the respiratory gas in another way. In particular, it can be a breathing regulator and/or a breathing tube with at least one valve. In particular, the gas-conducting element can be an element which has at least one tube and/or at least one different type of gas-conducting device with an interior space and/or at least one valve in which cleaning fluid should generally not be applied. For example, the gas-conducting element can be a region of the breathing apparatus that, during operation, is charged with a pressure above the normal pressure, for example a gas-conducting region of a breathing apparatus above a normal pressure, for example above 1.5 bar, in particular above 2 bar. In particular, this can be a respiratory-gas-conducting region of a breathing regulator, wherein the breathing regulator can in principle be a single-stage or else a multi-stage breathing regulator. The gas-conducting element here can be, for example, part of a first stage and/or a second stage and/or optionally further stages of the breathing regulator or else a complete breathing regulator. For example, the gas-conducting element can be a region between a tube connection and a valve of a breathing regulator, or the gas-conducting element can comprise such a region.

The term “basket rack”, as is used here, is a broad term which is to be given its customary and common meaning, as understood by a person skilled in the art. The term is not restricted to a specific or adapted meaning. The term can refer, without restriction, in particular to a mechanical basic structure of the cleaning basket, which basic structure can in particular have a basket base, and additionally one or more basket walls which can in particular extend from the basket base. The basket rack can be produced in particular entirely or partially from at least one material, selected from the group consisting of a plastic and metal. The basket rack can be configured, for example, entirely or partially as a wire rack, for example as a wire grating. Alternatively or additionally, the basket rack can also be entirely or partially configured as a plastics rack, for example as a plastics rack with a plurality of openings, for example as a plastics grating. Overall, the basket rack can have, for example, one or more openings so that cleaning fluid can flow out of an interior of the basket rack. The basket rack can, for example, entirely or partially have a cuboidal basic structure, for example with a rectangular supporting surface.

The term “pressure connection”, as used here, is a broad term which is to be given its customary and common meaning, as understood by a person skilled in the art. The term is not restricted to a specific or adapted meaning. The term can, without restriction, refer in particular to basically any connecting element to which the at least one gas-conducting element can be connected in order to provide compressed gas from the pressurization device to the at least one gas-conducting element. A connection between the pressure connection and the gas-conducting element can be understood in general as meaning a fluidic connection, in particular a gas connection, such that the fluid under pressure of the pressurization device can be transferred into the gas-conducting element of the breathing apparatus, in particular into an interior space of the gas-conducting element, preferably without a fluid loss and/or pressure loss occurring in this connection. The pressure connection can comprise at least one mechanical connecting element, in particular a positive and/or non-positive connection, so that the breathing apparatus, in particular the gas-conducting element, can be fixedly connected to the pressure connection. For example, it can be a screw connection and/or a clamping connection and/or a tensioning connection, for which purpose the pressure connection and/or the breathing apparatus can in each case have at least one mechanical connecting element. It can be, in particular, a plug-in connection in the form of a quick-action coupling and/or a thread, for example a coupling piece for a respiratory connection of a breathing regulator. A quick-acting coupling should be understood in this case in general as meaning a gas-tight and/or liquid-tight plug-in connection between two fluid-conducting components, which plug-in connection can be mechanically secured by a mechanical fixing that can be produced and released in a simple and quick way, in particular without use of a screw closure, for example by at least one clip and/or a bayonet closure and/or a screw closure, for example a union nut. The connection can in particular be produced without a tool. In particular, the pressure connection can comprise a plurality of adaptors of a fixed or exchangeable design for connection to different types of gas-conducting elements. By means of these adaptors, a plurality of different gas-conducting elements can be connectable directly or indirectly to the pressure connection, for example different kinds and/or different types of gas-conducting elements, for example of different makes or manufacturers. For example, a set of adaptors with a plurality of different coupling pieces (for example coupling pieces of a quick-action coupling) and/or threads can be provided for the connection of different gas-conducting elements. For example, they can be different quick-action coupling systems and/or standard threads. Alternatively or in addition, the at least one pressure connection can, however, also be configured as a fixed pressure connection for a certain type of connecting element or for a certain type of gas-conducting element. It is particularly advantageous if the pressure connection and/or the coupling system thereof is configured in such a manner that, firstly, gas cannot escape if no counterpart is connected, and that, in this case, no other medium, for example cleaning fluid, can enter the gas-conducting element either.

The pressure connection can be connected or connectable directly or indirectly to the pressurization device. As is described in more detail below, there are a plurality of possibilities in respect of the configuration of the pressurization device. For example, it can be entirely or partially integrated in the cleaning basket, for example in the form of at least one compressed gas cylinder. In this case, for example, there can be a compressed gas line between the integrated pressurization device and the pressure connection in the cleaning basket. At least one valve, for example, can be provided in said compressed gas line.

Alternatively or additionally, the pressurization device can also be, for example, entirely or partially accommodated in a cleaning device, into the cleaning chamber of which the cleaning basket is introduced, in the form of a pressurization device integrated in the cleaning device and/or in the form of a connection of the cleaning device, for example, to an on-site compressed gas network or an on-site compressed gas line. In this case, for example, the cleaning basket can be connectable to the pressurization device via at least one compressed gas line. Said compressed gas line can be, for example, attached automatically when the cleaning basket is inserted into the cleaning chamber, as will be described in more detail below. Alternatively or additionally, however, the compressed gas line may also be attached, for example, manually, for example manually to a corresponding connection on the cleaning basket when the cleaning basket is introduced into the cleaning chamber of the cleaning device. A cleaning system which comprises the cleaning basket and the cleaning device and which will be described in more detail below can therefore also comprise the at least one compressed gas line which connects the pressurization device and the pressure connection. The compressed gas line can be entirely or partially a component part of the cleaning basket or else can also be entirely or partially a component part of a further part of the cleaning system, for example of the cleaning device, or a separate component.

The term “pressurization device”, as is used here, is a broad term which is to be given its customary and common meaning, as understood by a person skilled in the art. The term is not restricted to a specific or adapted meaning. The term can, without restriction, refer in particular to a device which is configured in order to be able to provide a fluid, in particular a gas, with a pressure above the normal pressure, for example a pressure of at least 1.5 bar, preferably a pressure of at least 2 bar. The pressurization device is configured to charge the gas-conducting element with compressed gas. A compressed gas can be understood in general as meaning any desired gas which has a pressure above standard pressure, that is to say a pressure of above 1 bar. It can be, in particular, a pressure above 1.5 bar, in particular above 2 bar, and particularly preferably above 3 bar. In particular, the pressurization device can be configured in such a manner that the pressurization with the compressed gas takes place such that all the cleaning fluid is kept away from an interior space of the breathing apparatus that is charged with the compressed gas. The compressed gas can be, for example, compressed air or some other gaseous medium at a positive pressure, for example nitrogen, carbon dioxide or the like. In particular, an inert gas can be used as the compressed gas. The cleaning device can, in particular, be configured in such a manner that the pressurization via the pressurization device takes place during at least one cleaning operation, for example during at least one program step of a single-step or multi-step cleaning program. In particular, the charging with the compressed gas can take place at the same time as the cleaning fluid is applied to the breathing apparatus. The pressurization device can in particular have at least one valve, in particular at least one valve which can be activated by a controller of the cleaning device.

The pressurization device can in particular have at least one element selected from the group consisting of:
at least one external pressure connection for connecting to an external compressed gas source, in particular an on-site compressed gas line and/or a compressed gas cylinder, to the cleaning device; and/or
a compressed gas source which is integrated in the cleaning device, in particular an integrated compressed gas cylinder and/or an integrated compressor; and/or
a compressed gas source which is integrated in the cleaning basket, in particular an integrated compressed gas cylinder and/or an integrated compressor.

As explained above, the pressure connection can comprise in particular at least one positive and/or non-positive connecting element. This connecting element can be configured to produce a mechanical connection to the gas-conducting element in addition to the gas-tight connection. In particular, said at least one connecting element can have at least one quick-acting coupling and/or at least one thread. In general, the pressure connection can be entirely or partially integrated in the cleaning basket. The cleaning basket can in general have in particular at least one releasable compressed gas connection to the cleaning device such that the compressed gas can be provided to the cleaning basket via the cleaning device, for example when the pressurization device is entirely or partially integrated in the cleaning device and/or when the pressurization device entirely or partially comprises an external pressure connection which is connected to the cleaning device. The releasable compressed gas connection between the cleaning basket and the cleaning device can be closed, for example, manually and/or automatically when the cleaning basket is introduced into the cleaning chamber and/or can be manually or automatically decoupled when the cleaning basket is removed from the cleaning chamber. The cleaning basket and the cleaning device can thus have, for example, corresponding compressed gas connecting parts which are couplable, in particular reversibly, to one another.

The pressurization device is configured to charge the gas-conducting element with compressed gas. A compressed gas can be understood in general as meaning any desired gas which has a pressure above normal pressure, that is to say a pressure above 1 bar. It can be, in particular, a pressure above 1.5 bar, in particular above 2 bar, and particularly preferably above 3 bar. Particularly preferably, the pressurization device is configured in such a manner that the pressurization with the compressed gas takes place such that all the cleaning fluid is kept away from an interior space of the breathing apparatus that is charged with the compressed gas. The compressed gas can be, for example, compressed air or some other gaseous medium at a positive pressure, for example nitrogen, carbon dioxide or the like. In particular, an inert gas can be used as the compressed gas. The cleaning device can, in particular, be configured in such a manner that the pressurization via the pressurization device takes place during at least one cleaning operation, for example during at least one program step of a single-step or multi-step cleaning program. In particular, the charging with the compressed gas can take place at the same time as the cleaning fluid is applied to the breathing apparatus. The pressure connection can accordingly be arranged in particular within the cleaning chamber such that the charging with compressed gas and application of cleaning fluid can take place at the same time.

As explained above, the cleaning basket has at least one pressure sensor which is configured to identify a pressurization of the pressure connection. The term “pressure sensor”, as is used here, is a broad term which is to be given its customary and common meaning, as understood by a person skilled in the art. The term is not restricted to a specific or adapted meaning. The term can, without restriction, refer in particular to basically any desired element or a device configured to detect the pressurization qualitatively or quantitatively. The pressure sensor can make use, for example, of measurement principles which are basically known to a person skilled in the art. For example, the pressure which is to be detected can bring about a change in at least one part of the pressure sensor, for example at least one change selected from the group consisting of a displacement, a change in position, a change in orientation, a deformation, an extension or a rotation, which can be detected qualitatively or quantitively, for example by means of at least one measurement principle selected from the group consisting of a mechanical measurement principle, an optical measurement principle and an acoustic measurement principle. The pressurization by the pressure sensor can be identified qualitatively or quantitatively, as explained above. A combination of qualitative and quantitative detection is also conceivable, for example if a plurality of pressure sensors are included. A qualitative detection can be undertaken, for example, to the effect that it is determined whether at least one predetermined minimum pressure is or is not present, wherein the minimum pressure can be fixedly predetermined or else can be adjustable variably, for example by a user and/or automatically by the cleaning device, for example in order to permit adaptation to a cleaning program or to a cleaning program step. A quantitative detection can be undertaken, for example, to the effect that the pressure sensor generates at least one detectable measurement variable, for example at least one measurement signal, which correlates with the prevailing pressure. This measurement signal can also be or comprise, for example, a position of at least one measurement member or at least one indicator of the pressure sensor, said position at least partially correlating with the pressure.

The result of the identification of the pressurization can also be configured in accordance with these possibilities of configuring the pressure sensor. This result can thus comprise at least one item of information which describes the pressurization qualitatively and/or quantitatively. For example, the item of information can comprise a binary or Boolean item of information which indicates whether the pressurization reaches or does not reach the above-described at least one minimum value. “Reached” can be understood here as meaning “is greater than” or else “is at least the same size as”. By contrast, a quantitative item of information can comprise, for example, at least one measurement value or another measurement variable or the transmission variable thereof which can assume more than two values and which, for example quantitatively via at least one measurement range and/or in at least three stages, describes which value the pressurization assumes. The item of information can be present in various ways, for example in at least one form selected from the group consisting of: a mechanical form, for example a position of a mechanical indicator; a magnetic item of information, for example an orientation or other configuration of a magnetic field; an electrical item of information, for example an analogue or digital electrical item of information; an optical item of information, for example a color, intensity or position of at least one optical element.

As also explained above, the cleaning basket has at least one transmission device for transmitting at least one result of the identification of the pressurization to a cleaning device. The term “transmission device”, as is used here, is a broad term which is to be given its customary and common meaning, as understood by a person skilled in the art. The term is not restricted to a specific or adapted meaning. The term can, without restriction, refer in particular to a device which is basically as desired and by means of which at least one item of information can be transmitted, in particular from one device to another device. The configuration of the transmission device can be adapted in accordance with the above-described possibilities when configuring the result of the identification of the pressurization. Accordingly, the transmission device can have, for example, at least one interface selected from the group consisting of a mechanical, a magnetic, an electrical and an optical interface.

In particular, the pressure sensor can have at least one movable element, i.e. at least one element which can change at least one position and/or orientation, for example into two or more positions, stepwise or continuously. This movable element can be movable in particular by means of the pressurization. The movable element can thus be equipped with at least one hydraulic surface or can be connected thereto, on which hydraulic surface the pressure of the pressurization device can act. In particular, the movable element can be movable by means of the pressurization counter to a restoring force. Said restoring force can comprise, for example, gravity and/or at least one elastic restoring force, for example a spring force. The at least one movable element can have, for example, at least one element selected from the group consisting of a spring-loaded piston and a gravity-restoring piston. However, other types of movable elements which can act as indicators of the pressurization are also conceivable, as a person skilled in the art will recognize. In particular, the movable element can have at least one magnetic element. This magnetic element can be, for example, a component part of the abovementioned piston or can be coupled to the latter. The configuration of the pressure sensor with the movable or movably mounted element is a particularly simple and robust solution which is in particular also suitable for the special conditions within a cleaning chamber, for example the high air humidity. The solution is less susceptible to faults, easy to implement and is insensitive, for example, to vapor and changing atmospheric conditions in the cleaning chamber.

The cleaning basket can furthermore have, in addition to the transmission device or else as a component part of same, at least one indicator which is configured to visually display a result of the identification of the pressurization of the pressure connection. This indicator can be, for example, a color indicator, for example a color indicator which changes at least one position of at least one colored element in accordance with the result of the identification of the pressurization. For example, a colored piston can be pressed out of a housing by pressurization, which can be seen visually. Other possibilities are also conceivable.

As explained above, the cleaning basket can furthermore have at least one mask holder for holding at least one protective breathing mask in the cleaning basket. The term “mask holder”, as is used here, is a broad term which is to be given its customary and common meaning, as understood by a person skilled in the art. The term is not restricted to a specific or adapted meaning. The term can, without restriction, refer in particular to a device which is configured to hold at least one protective breathing mask, in particular during a cleaning operation. The mask holder can in particular have at least one projection which extends from the cleaning basket and in particular from a base of the cleaning basket and/or a wall of the cleaning basket, for example into an interior space of the cleaning basket and/or upward from the cleaning basket, and which is configured to hold and/or to fix the at least one protective breathing mask, in particular at a distance from a base of the cleaning basket. The mask holder can be produced, for example, entirely or partially from at least one plastic and/or entirely or partially from at least one metallic material. The mask holder can also be entirely or partially connected to the basket rack or else formed integrally with the basket rack.

Furthermore, the cleaning basket can have at least one suction element which has at least one suction opening and is configured to extend into the at least one protective breathing mask held by the mask holder. The suction element can be configured to be charged with negative pressure and to suck cleaning fluid out of at least one interior space of the protective breathing mask into the suction opening. The term “suction element”, as is used here, is a broad term which is to be given its customary and common meaning, as understood by a person skilled in the art. The term is not restricted to a specific or adapted meaning. The term can, without restriction, refer in particular to a device or an element configured to remove at least one fluid from a specified region by the suction operation, in particular by application of a negative pressure. In particular, the suction element can have at least one element selected from the group consisting of a pipe, a tube and a channel, wherein the element has at least one opening through which fluid can enter the element, and wherein the element itself can be charged with a negative pressure. For example, the element can have at least one connection to at least one negative pressure source or vacuum source or for the connection of at least one negative pressure source or vacuum source, wherein the connection is connected fluidically to the at least one opening, which can also be referred to as a suction opening.

Accordingly, the term “suction opening”, as is used here, is a broad term which is to be given its customary and common meaning, as understood by a person skilled in the art. The term is not restricted to a specific or adapted meaning. The term can, without restriction, refer in particular to an opening in a wall of the suction element, through which opening fluid can enter an interior space, for example into a lumen, which can be charged with a negative pressure, of the suction element. The opening can in principle have any desired cross section, for example a round, oval or polygonal cross section. For example, one single opening or a plurality of openings can be provided in the wall of the suction element. For example, the at least one suction opening can comprise at least one passage opening in at least one wall of a pipe, a tube or a channel of the suction element.

The suction element can be entirely or partially connected to the mask holder and/or integrated in the mask holder. Accordingly, the suction element can also be formed, for example, as a component part of the mask holder and/or can be connected fixedly or releasably to the mask holder. Alternatively or additionally, the suction element can, however, also be connected entirely or partially fixedly or releasably to other component parts of the cleaning basket or can be entirely or partially integrated in other component parts of the cleaning basket. The suction element can thus also be connected to the basket rack. A fixed or else a releasable connection is conceivable here. For example, the suction element can be configured to be connected releasably to one or a plurality of other component parts of the cleaning basket, for example to the basket rack or to the mask holder, and to be removed, for example, for cleaning purposes.

As explained above, the suction element is configured to extend into the protective breathing mask held by the mask holder. Accordingly, the suction element can in particular have an elongate element, for example an element, the longitudinal extent of which exceeds its lateral extent by at least a factor of 2, for example by at least a factor of 3. In particular, the suction element can accordingly be configured, for example, as a tube or pipe or else as a loop of a tube or a pipe, in order to extend into at least one interior space of the protective breathing mask. In terms of space, the suction element can be positioned in particular in such a manner that it protrudes out of the mask holder or protrudes from the mask holder such that, when the protective breathing mask is accommodated on the mask holder, the suction element extends into an interior space which is formed, for example, by a wall of the protective breathing mask, which wall faces the mask holder. The suction element can be configured here to be rigid or else, preferably, flexible, as will be explained in more detail below, and therefore the suction element can be introduced, for example, flexibly into the interior space and, for example, can be introduced specifically into a space at a seal of the protective breathing mask in order to suck off cleaning fluid there.

Furthermore, as explained above, the suction element can be configured to be charged with negative pressure. As explained above, this charging with negative pressure and thus the suction operation while the at least one cleaning fluid is applied to the breathing apparatus or else after the cleaning fluid has been applied can take place, for example, during a drying step or before a drying step. By means of the charging of the suction element with negative pressure, cleaning fluid is sucked out of the at least one interior space of the protective breathing mask into the suction opening. For this purpose, the suction element, as explained above, can have, for example, at least one lumen which is surrounded, for example, by a wall of the suction element, for example a lumen of a pipe, of a tube or of a channel of the suction element. The suction opening here can penetrate, for example, the wall which surrounds the lumen, and therefore cleaning fluid can enter the lumen through the suction opening. The lumen can in particular be elongated. The lumen can be connected or connectable, for example, at at least one connecting point to at least one negative pressure source, also referred to as a vacuum source, for example at at least one port or at a different type of connecting point.

Further advantageous refinements can refer to the transmission device. Various possibilities for configuring the transmission device have already been mentioned above. In particular, the transmission device can have at least one device selected from the group consisting of: a magnetic transmission element, in particular a movably mounted magnetic element which is changeable in a position by means of the pressurization; a mechanical actuating element, in particular a movably mounted mechanical element which is changeable in a position by means of the pressurization; an optical element, in particular a movably mounted optically detectable element which is changeable in a position by means of the pressurization; a magnetic transmission element, in particular a movably mounted magnetic element which is changeable in a position by means of the pressurization; a mechanical actuating element, in particular a movably mounted mechanical element which is changeable in a position by means of the pressurization; an optical element, in particular a movably mounted, optically detectable element which is changeable in a position by means of the pressurization; an acoustic element; an electronic element; an electromagnetic element, in particular an electromagnetic element which is configured to generate and/or to change an electrical signal by means of the pressurization; an electronic element; a mechanical-electrical transducer, in particular an element which is configured to convert a pressurization into an electrical signal, in particular a mechanical-electrical transducer selected from the group consisting of an element which is based on the use of a strain gauge and, by means of the pressurization, can change an electrical resistance in an electrical conductor, an element which, on the basis of crystalline structures, can change an electrical resistance in an electrical semiconductor by means of the pressurization, or an element which, on the basis of crystalline structures, can generate an electrical voltage by means of the pressurization; a mechanical-acoustic transducer, in particular an element which, by means of the pressurization, can generate an acoustic signal, for example either directly and/or indirectly, in particular by an electrically acoustic generation of sound. The at least one pressure sensor can transmit the result of the identification of the pressurization in particular directly or indirectly and wirelessly to the cleaning device, for example by a direct pressure measurement in the cleaning basket with radio transmission to the cleaning device, for example via RFID and/or Bluetooth, in particular low-energy Bluetooth.

The at least one cleaning basket can furthermore have at least one energy source and/or at least one energy store, in particular for supplying energy to the at least one pressure sensor and/or the transmission device. For example, at least one battery and/or at least one rechargeable battery can be provided in the at least one cleaning basket. In turn, alternatively or additionally, the at least one cleaning basket can also have at least one electrical interface for the external energy supply. In turn, alternatively or additionally, the at least one cleaning basket can also have at least one energy generation device and/or at least one energy converter, for example at least one energy harvesting system, in particular a converter which is configured to convert vibrations, pressure, temperature or other forms of energy into electrical energy. In this way, the at least one pressure sensor and/or the at least one transmission device can be supplied with energy.

These possibilities of transmitting information are usable readily and without disturbance in particular in the hot and moist atmosphere of a cleaning chamber of the cleaning device. The described devices can be in particular entirely or partially connected, coupled or identical in terms of components to the above-described at least one movable element which is movable by means of the pressurization.

In a further aspect, a cleaning device for cleaning a breathing apparatus is proposed. The cleaning device comprises at least one cleaning chamber for receiving at least one cleaning basket proposed here, for example in accordance with one or more of the above-described configurations and/or in accordance with one or more of the configurations still to be described in more detail below. The cleaning device furthermore has at least one application device for applying at least one cleaning fluid to the breathing apparatus. Furthermore, the cleaning device has at least one interface for receiving the at least one result of the identification of the pressurization by the pressure sensor of the cleaning basket.

The term “cleaning device”, as is used here, is a broad term which is to be given its customary and common meaning, as understood by a person skilled in the art. The term is not restricted to a specific or adapted meaning. The term can, without restriction, refer in particular to a device which is configured to free items for cleaning from adhering macroscopic or microscopic impurities or to at least partially remove such impurities. In addition, a disinfecting effect can optionally be carried out. The cleaning device, as will be explained further below, can be configured in particular as a washer, in particular as a converted dishwasher. The washer can be configured here, for example, as a single-chamber washer, in particular as a commercial single-chamber washer with a multi-circuit rinsing system, as a front-loading machine or else as a hood-type washer, in particular as a pass-through washer. Dishwashers with an exchange of water, in particular what are referred to as single-circuit systems, are also usable. Alternatively or additionally to washers, different types of cleaning devices can also be used.

The cleaning device comprises, as explained above, at least one cleaning chamber. The term “cleaning chamber”, as is used here, is a broad term which is to be given its customary and common meaning, as understood by a person skilled in the art. The term is not restricted to a specific or adapted meaning. The term can, without restriction, refer in particular to a completely or partially closed chamber, within which the cleaning operation can be completely or partially carried out. The cleaning chamber can have in particular at least one housing which entirely or partially surrounds the cleaning chamber. A single cleaning chamber can be provided here, or in principle also a plurality of cleaning chambers can be provided, for example sequentially. The cleaning chamber can have, for example, at least one opening for loading the cleaning chamber with the item for cleaning. For example, this one opening can be provided with a flap arranged on a front side of the cleaning chamber and/or an upper side of the cleaning chamber. Alternatively, hoods for closing the cleaning chamber are also possible, for example within the scope of what are referred to as hood-type dishwashers or push-through washers. Other configurations are in principle also possible.

The cleaning chamber, as explained above, is configured for receiving the at least one cleaning basket, for example in accordance with one or more of the above-described configurations and/or in accordance with one or more of the embodiments still to be described in more detail below. For this purpose, the cleaning chamber can have, for example, at least one basket receptacle, for example at least one rail, into which the cleaning basket can be pushed, and/or at least one holding device or transport device, by means of which the basket can be transported into the cleaning chamber, out of the cleaning chamber or through the cleaning chamber.

The cleaning device furthermore has, as explained above, at least one application device for applying at least one cleaning fluid to the breathing apparatus. The application device can be configured in particular for applying the at least one cleaning fluid to the breathing apparatus in the cleaning chamber. The application device can have, for example, at least one tank for receiving the at least one cleaning fluid and, for example, one or more nozzles. In particular, the application device can have, for example, at least two nozzle arms received in the cleaning chamber. In addition, the application device can have further elements, such as, for example, one or more pumps for transporting the cleaning fluid to the at least one nozzle, and one or more pipelines. The application device can be controlled in particular by at least one controller of the cleaning device.

The term “application device”, as is used here, is a broad term which is to be given its customary and common meaning, as understood by a person skilled in the art. The term is not restricted to a specific or adapted meaning. The term can, without restriction, refer in particular to basically any desired device by means of which the at least one cleaning fluid, in particular the at least one cleaning liquid, can be applied to the items for cleaning within the cleaning chamber. The application device has, for example, at least one nozzle system with at least one nozzle, for example with one or more nozzle arms which can be arranged, for example, above and/or below the item for cleaning. Furthermore, the application device can comprise at least one pump and at least one conducting system, for supplying cleaning liquid to the nozzle system. For example, a nozzle system and a line system for applying with cleaning liquid from a tank, and also at least one corresponding pump, can be provided. Alternatively or additionally, for example, at least one nozzle system can also be directly supplied from a feed line without a pump being required for this purpose. The cleaning device can be configured in particular as a cleaning device with two tanks, a washing tank and a rinsing tank. Accordingly, the application device can comprise in particular a washing system and a rinsing system, wherein, for example, the washing system can have the washing tank and a washing nozzle system and also a washing line system and a washing pump, for example a recirculating pump. The rinsing system can have, for example, a rinsing tank which is separate from the washing tank, and furthermore a rinsing nozzle system, a rinsing line system and optionally a rinsing pump. The rinsing nozzle system and the washing nozzle system can be formed, for example, entirely or partially separately. However, said nozzle systems can optionally also be entirely or partially combined, for example in a common housing.

As explained above, the application device can have in particular at least one nozzle. Said at least one nozzle can comprise in particular at least one nozzle selected from the group consisting of: a spray nozzle; a rinsing nozzle; a spray arm, in particular a rotatable spray arm; a nozzle which can be operated in the recirculating operating mode.

The term “cleaning fluid”, as is used here, is a broad term which is to be given its customary and common meaning, as understood by a person skilled in the art. The term is not restricted to a specific or adapted meaning. The term can, without restriction, refer in particular to a liquid which, upon impinging upon items for cleaning, can deploy a cleaning action. Accordingly, hereinafter, the term “cleaning liquid” instead of the term “cleaning fluid” is also used without restricting the use of other fluid media. In particular, the cleaning liquid can comprise an aqueous liquid, for example water and/or water with one or more additives, for example with one or more cleaning agent concentrates and/or rinse aids and/or disinfectants. Cleaning fluid can be applied to the breathing apparatus in such a manner that an individual cleaning liquid is used, or else in such a manner that a combination of a plurality of cleaning liquids is used. If a plurality of cleaning liquids are provided, the different cleaning liquids can be applied simultaneously or else sequentially. The cleaning basket with the breathing apparatus can remain, for example, in a stationary manner within a cleaning chamber of a cleaning device and the various cleaning liquids and/or cleaning fluids can be applied successively.

As furthermore explained above, the cleaning fluid can in particular comprise at least one cleaning fluid selected from the group consisting of: an aqueous cleaning fluid; a cleaning fluid with at least one cleaning agent solution; a cleaning fluid with at least one rinse aid; a cleaning fluid with at least one disinfectant; a post-rinsing fluid; demineralized water; and a heated cleaning fluid, in particular a cleaning fluid heated to a temperature of 30° C. to 70° C. and particularly preferably 60° C.

“Cleaning fluid is applied” can be understood here in general as meaning any desired bringing of the item for cleaning, in this case the breathing apparatus, into contact with the at least one cleaning fluid. This can take place, for example, in the form of a direct application, for example by sprinkling, dripping, spraying or a combination of the abovementioned and/or other direct types of application, in which the cleaning fluid impinges directly against the item for cleaning, for example the breathing apparatus and/or the breathing mask. This configuration of the application can take place in particular in the case of a fixed cleaning chamber. Alternatively or additionally, the application can also take place in such a manner that the cleaning chamber is completely or partially filled with cleaning fluid such that the items for cleaning which are accommodated in the cleaning chamber come into contact with the cleaning fluid at least in one position of the cleaning chamber. This configuration of the application can be used in particular in the case of a configuration of the cleaning chamber as a movable cleaning chamber, for example in the form of a drum. Combinations of the types of application mentioned and/or other types of application are also possible. Irrespective of whether the cleaning chamber is configured to be rigid or movable, the cleaning fluid can be applied in the simple operating mode by the cleaning fluid merely being applied once to the items for cleaning, for example the breathing apparatus and/or the breathing mask. Alternatively or additionally, however, cleaning can also take place in the circulating operating mode by cleaning fluid being applied repeatedly to the items for cleaning. Such circulating operating modes and circulating circuits are known, for example, from conventional washers.

The cleaning device can be configured, for example, in order to carry out at least one cleaning program by means of the application device, for example in a manner controlled by at least one controller of the cleaning device, in which the item for cleaning, for example the at least one cleaning basket with the breathing apparatus, is accommodated in a stationary manner in the cleaning chamber and cleaning fluid is applied in differing ways successively in one or more cleaning steps of the cleaning program, for example first of all in at least one washing step with the at least one washing fluid and in at least one rinsing step with the at least one rinsing fluid. Furthermore, at least one drying step can be provided which is, for example, mounted downstream of the application of the cleaning fluid in the one cleaning chamber when the items for cleaning are accommodated in a stationary manner. Accordingly, the cleaning device can be configured in particular as an automatic programmed washer. Alternatively, however, the cleaning device can also be configured, for example, as a transport cleaning device, in which the cleaning basket with the breathing apparatus is transported through one or more cleaning zones.

The term “tank”, as is used here, is a broad term which is to be given its customary and common meaning, as understood by a person skilled in the art. The term is not restricted to a specific or adapted meaning. The term can, without restriction, refer in particular to a completely or partially closed vessel which is configured for receiving at least one fluid, in particular at least one liquid. As explained above, the at least one tank can have, for example, at least one washing tank. Said at least one washing tank can be arranged, for example, in a base of the cleaning chamber or else can be fluidically connected to a base of the cleaning chamber such that cleaning fluid can flow out of the cleaning chamber into the washing tank. For example, the washing tank can be separated from the cleaning chamber by at least one strainer. In addition to the at least one washing tank, the at least one tank can also furthermore comprise one or more further tanks. The at least one tank can thus have, for example, at least one rinsing tank which, for example, can be formed separately from the washing tank. For example, at least one rinsing fluid can be treated, for example temperature-controlled, in the at least one rinsing tank while a washing step is still proceeding in the cleaning chamber. The rinsing tank can comprise, for example, at least one boiler.

The cleaning device furthermore has at least one interface for receiving the at least one result of the identification of the pressurization by the pressure sensor of the cleaning basket. The term “interface”, as is used here, is a broad term which is to be given its customary and common meaning, as understood by a person skilled in the art. The term is not restricted to a specific or adapted meaning. The term can, without restriction, refer in particular to a device which is basically configured as desired and which is configured to receive at least one item of information and then to correspondingly optionally completely or partially process and/or transmit it, for example to at least one controller. The interface can be configured in particular as a counterpart to the above-described transmission device, and therefore the interface can be configured in particular to receive an item of information provided by the transmission device. Accordingly, with regard to possible configurations of the information transmission, reference can be made to the above-described possibilities, wherein a person skilled in the art will recognize that a corresponding device for receiving information in the interface can be assigned to one specific possibility of transmitting information by the transmission device. In particular, the interface can have at least one element selected from the group consisting of: a magnetic interface, in particular an interface with at least one magnetic sensor for detecting at least one magnetic field; a mechanical interface, in particular a mechanical interface with at least one mechanical sensor, in particular a mechanical sensor for detecting at least one position of a mechanical activating element; an optical interface, in particular an optical interface with at least one optical sensor for optically detecting at least one position of at least one optically detectable element; at least one electrical interface, for example an electrically conductive connection; at least one radio interface, in particular an interface which is suitable for using the RFID technology; an acoustic interface, for example a microphone; an electronic interface. In particular, the interface can have at least one Reed sensor, i.e. a magnetic switching element. The Reed sensor can be configured in particular to detect at least one position of at least one movably mounted magnetic element of the cleaning basket which is changeable in a position by means of the pressurization.

The cleaning device can furthermore comprise at least one controller which is configured to control at least one cleaning program in the cleaning device. The term “controller”, as is used here, is a broad term which is to be given its customary and common meaning, as understood by a person skilled in the art. The term is not restricted to a specific or adapted meaning. The term can, without restriction, refer in particular to a single-part or multi-part device of the cleaning device, which is configured to completely or partially control and/or to regulate an operation of the cleaning device. In particular, the controller can be configured to change, in particular to control and/or to regulate, one or more operating parameters of the cleaning device, for example at least one temperature, at least one pressure, at least one pump output, at least one valve position, at least one heating output or else a combination of two or more of the operating parameters mentioned. The controller can comprise in particular at least one data processing device, for example at least one processor. The controller can be configured in particular in terms of programming, for example to control or to carry out at least one cleaning program of the cleaning device. Accordingly, the controller facility can be partially realized by hardware or/also, alternatively or additionally, completely or partially by software. Furthermore, the controller can comprise at least one volatile and/or non-volatile data memory. Furthermore, the controller can comprise at least one interface, for example a human/machine interface for inputting commands and/or for outputting information, and/or a wireless or wired interface for unidirectional or bidirectional exchange of data and/or commands between the cleaning device and at least one further device. The controller can comprise in particular at least one computer and/or at least one processor. The controller can be in particular a central or decentralized machine controller of the cleaning device.

The controller can be configured in particular, for example configured by programming, in at least one program step of the cleaning program, to activate charging of the gas-conducting element of the breathing apparatus with at least one compressed gas, while the at least one cleaning fluid can be applied to the breathing apparatus at the same time. Furthermore, the controller can be configured, in at least one program step, to activate charging of the suction element with negative pressure such that penetrating cleaning fluid is sucked out of the at least one interior space of the protective breathing mask at least partially into the suction opening.

The controller can be configured in particular to receive the result of the identification of the pressurization by the pressure sensor of the cleaning basket from the interface. This can take place by the fact that the interface is entirely or partially a component part of the controller, and/or by the fact that the interface has an exchange with the controller, for example is connected via at least one data line and/or at least one signal line or else wirelessly. The result of the identification of the pressurization by the pressure sensor of the cleaning basket can be transmitted from the interface unchanged to the controller or, for example, also following modification, conversion or preprocessing.

As explained above, the cleaning device can furthermore have at least one pressurization device. In other words, the pressurization device can be entirely or partially a component part of the cleaning device. For possible configurations and options, reference can be made to the above description of the cleaning basket. The pressurization device can be connectable to the cleaning basket and can be configured to charge the at least one gas-conducting element of the breathing apparatus with compressed gas via the pressure connection of the cleaning basket while the at least one cleaning fluid is being applied to the breathing apparatus.

The cleaning device can furthermore have at least one further pressure sensor, i.e. at least one further pressure sensor in addition to the at least one pressure sensor of the cleaning basket. The at least one further pressure sensor, which can be a component part of the cleaning device, can be configured to monitor the pressurization by the pressurization device. Therefore, in addition to the detection by means of the at least one pressure sensor in the cleaning basket, at least one further monitoring of the pressure can take place in the cleaning device, and therefore, for example, an entire pressurization system comprising pressure-conducting parts of the cleaning device and pressure-conducting parts of the cleaning basket can be configured such that the pressure is monitored.

The controller of the cleaning device can be configured to receive at least one signal from the further pressure sensor. This signal can be, for example, an electrical signal which can be transmitted to the controller from the at least one further pressure sensor in wired form or else wirelessly, for example. It can be an analogue or else a digital data signal.

The controller can be configured, for example, to evaluate the signal, in particular by a corresponding configuration by programming and/or also by a corresponding electrical circuit. The controller can thus be configured in particular to identify a connection operation of a pressure connection of a cleaning basket to the pressurization device on the basis of a pressure fluctuation in the signal, in particular a sudden, temporally limited pressure drop. The controller can be configured in particular in order, in accordance with the identification of the connection operation, to undertake one or more certain actions, for example to enable a cleaning operation and/or to output a warning to a user of the cleaning device.

In general, the controller can be configured in particular so as, in accordance with the result of the identification of the pressurization, by means of the at least one pressure sensor of the cleaning basket and optionally additionally also by means of the at least one further optional pressure sensor of the cleaning device, to carry out at least one action selected from the group consisting of:
enabling the application of the at least one cleaning fluid to the breathing apparatus, in particular upon identification of a pressurization of the pressure connection, in particular upon identification of a pressurization of the pressure connection with a minimum pressure;
blocking the application of the at least one cleaning fluid to the breathing apparatus, in particular if pressurization of the pressure connection is not identified, in particular if pressurization of the pressure connection with a minimum pressure is not identified;
enabling at least one cleaning program of the cleaning device, in particular upon identification of pressurization of the pressure connection, in particular upon identification of pressurization of the pressure connection with a minimum pressure;
blocking at least one cleaning program of the cleaning device, in particular if a pressurization of the pressure connection is not identified, in particular if a pressurization of the pressure connection with a minimum pressure is not identified;
closing at least one opening device of the cleaning device, in particular closing with a time delay, in particular closing upon identification of a pressurization of the pressure connection, in particular upon identification of a pressurization of the pressure connection with a minimum pressure, wherein an automatic starting of at least one cleaning program after the closing, in particular the time-delayed closing, is also possible;
outputting at least one item of information about the pressurization of the pressure connection to a user of the cleaning device, in particular outputting a warning indication, in particular when, if a pressurization of the pressure connection with a minimum pressure is not identified, application of the at least one cleaning fluid to the breathing apparatus is intended to be started;
recording at least one item of information about the pressurization of the pressure connection, in particular recording a warning and/or when, after a warning, a cleaning program is started, in particular recording by means of the at least one controller;
blocking the cleaning device depending on at least one item of information about the pressurization of the pressure connection, in particular blocking a start of a cleaning program if the minimum pressure is fallen short of;
automatically starting at least one cleaning program of the cleaning device, in particular upon identification of a pressurization of the pressure connection with a minimum pressure.

Furthermore, the controller can be configured to record cleaning operations in the cleaning device. For example, data records about the cleaning operations in the cleaning device can be produced and optionally stored in at least one volatile or non-volatile data memory of the cleaning device and/or can be transmitted via at least one data interface to at least one further device, for example to at least one external computer. For example, the records can each store one or more operating parameters of the cleaning operations and furthermore other items of information, in particular times, and items of information about the item for cleaning or about a user. Furthermore, the recording can comprise the respective identification of the pressurization by the pressure sensor of the cleaning basket and optionally also the signal from the further pressure sensor or an item of information derived therefrom about the pressurization by means of the pressurization device. For example, it can be understood by evaluating the records whether the pressurization has or has not taken place, and in particular whether and/or if a rinsing program has then been started despite pressurization being absent.

The cleaning device can be configured in particular to use at least two different types of cleaning basket. The cleaning device can be configured, for example, to automatically identify the type of cleaning basket which specifically is received or is intended to be received in the cleaning chamber. The controller can be configured in particular to adapt at least one cleaning program to the type of cleaning basket identified. In particular, the cleaning device can be configured to differentiate between at least two different types of cleaning basket, in particular cleaning baskets with a pressure connection and cleaning baskets without a pressure connection. To identify the type of cleaning basket, the cleaning device can have in particular at least one basket identification device, the basket identification device being configured to identify the at least one cleaning basket in the cleaning chamber. The presence of the cleaning basket and/or also the type of cleaning basket can thus be identified automatically. The basket identification device can be configured in particular to differentiate between at least two different types of cleaning basket, in particular cleaning baskets with a pressure connection and cleaning baskets without a pressure connection. The basket identification device can be configured, for example, to directly identify the type of cleaning basket or a part of same, for example regarding a certain shape of the cleaning basket, which shape can be identified in particular optically or else mechanically. In the event of a mechanical identification, the cleaning basket can have, for example, a geometrical formation which, when present, directly actuates a switch in the cleaning chamber and/or leaves the switch unactuated if the formation is absent. The switch can also be actuated indirectly, for example, by, for example, a magnet in the geometrical formation actuating or not actuating a Reed switch in the wall of the rinsing chamber.

Alternatively or additionally, however, the at least one cleaning basket can also have at least one identifier which describes the type of cleaning basket or allows a conclusion to be drawn as to the type of cleaning basket. The at least one identifier can have, for example, at least one identifier selected from the group consisting of: an optical identifier, in particular a barcode and/or a QR code; a magnetic identifier, in particular a magnetic strip or another type of magnetic coding; an electrical identifier, in particular an identification chip. The basket identification device can have in particular a corresponding reading device for identifying and/or reading the at least one identifier.

In a further aspect, a cleaning system for cleaning a breathing apparatus is proposed. The cleaning system comprises at least one cleaning basket proposed here, for example according to one or more of the above-described configurations and/or according to one or more of the embodiments still to be described below. Furthermore, the cleaning system comprises at least one cleaning device proposed here, for example according to one or more of the above-described configurations and/or according to one or more of the embodiments still to be described below.

The term “cleaning system”, as is used here, is a broad term which is to be given its customary and common meaning, as understood by a person skilled in the art. The term is not restricted to a specific or adapted meaning. The term can, without restriction, refer in particular to a system, i.e. to an entirety of at least two functionally interacting components, said system serving for the cleaning within the meaning of the above definition. With regard to possible definitions and configurations of the cleaning basket and of the cleaning device, reference can be made to the corresponding above or following descriptions, definitions and options of said system components in the above description or else in the description below.

In a further aspect, a method for cleaning a breathing apparatus is proposed. The method comprises the use of at least one cleaning system proposed here, for example according to one or more of the above-described configurations and/or according to one or more of the embodiments still to be described below. Furthermore, the method comprises the steps mentioned in more detail below. These steps can be carried out in the sequence mentioned. However, a different sequence is basically also possible. Furthermore, two or more of the method steps mentioned can be carried out overlapping in time or simultaneously. Furthermore, one or more of the method steps mentioned can be carried out once or else repeatedly. Beyond the steps mentioned, the method can comprise further method steps which are not mentioned. The method comprises the following steps:
a) introducing the breathing apparatus into the cleaning basket;
b) connecting at least one gas-conducting element of the breathing apparatus to the pressure connection of the cleaning basket;
c) charging the gas-conducting element with compressed gas;
d) introducing the cleaning basket into the cleaning chamber of the cleaning device;
e) identifying the pressurization by means of the pressure sensor of the cleaning basket; and
f) transmitting the result of the identification of the pressurization to the cleaning device.

For possible configurations of said method steps, reference can be made to the above description of the system components of the cleaning system.

The method can furthermore comprise the following step:
g) checking the result of the identification of the pressurization and, if the result reveals that the gas-conducting element has been charged with compressed gas, applying at least one cleaning fluid to the breathing apparatus.

Accordingly, as explained above, the controller can be configured, for example, only to enable the entire cleaning program and/or the at least one program step in which the at least one cleaning fluid is applied to the breathing apparatus, if the result of the identification of the pressurization indicates that the gas-conducting element has been charged with compressed gas. This enquiry or condition can be, for example, also part of the method only if the cleaning device and/or the basket identification device have/has identified that the cleaning basket in the cleaning chamber is a cleaning basket with a pressure connection and not, for example, a cleaning basket without a pressure connection, in particular for cleaning other component parts of personal protective equipment.

The method can furthermore comprise the following step:
h) monitoring a pressurization of the pressure connection of the cleaning basket by a pressurization device of the cleaning device by means of at least one further pressure sensor of the cleaning device.

The enquiry in step g) above can also be analogously transmitted to the result of step h). Thus, the result of the monitoring can likewise enter the enabling of the application of at least one cleaning fluid to the breathing apparatus, for example as an additional condition. This can also take place, for example, in turn depending on whether a cleaning basket with a pressure connection has or has not been identified.

The cleaning basket, the cleaning device, the cleaning system and the method have numerous advantages over known devices and methods of the type mentioned. In particular, the above-described disadvantages of known devices and methods can be at least substantially avoided. In general, the reliability of the cleaning operation of safety-relevant protective breathing equipment parts can be significantly increased by means of the present invention. Incorrect operations, for example due to missing or incorrect connection, in particular of the compressed gas line to the cleaning basket, can thereby be avoided. A failure of the pressurization device in some other way, in one or more of the above-described possibilities, and other technical defects can also be identified in this way and appropriate measures initiated. For example, an interruption to an on-site supply of compressed air and/or a failure of a valve in the compressed gas supply, for example a compressed air magnetic valve, can be identified. The controller can take appropriate measures, for example stopping or preventing a cleaning operation and/or displaying a warning or error message indicating the problem which is present.

Technically, the proposed solution can be realized comparatively simply, by simple modifications of the cleaning basket and/or of the cleaning device. For example, baskets relevant to compressed air, i.e. cleaning baskets with a pressure connection, can be equipped with a spring- or gravity-restoring piston which can displace the position of a magnet when compressed gas is applied, in particular when compressed air is applied, for example in tubes and/or piping of the cleaning basket. A correspondingly positioned receiver of the interface of the cleaning device, for example a Reed switch in the cleaning device, for example the washer, can identify the displaced position of the magnet as an indicator of the compressed air applied and can therefore indicate to the controller, for example, to enable starting the cleaning operation. The item of information about the successful or existing compressed gas connection, for example the Reed switch signal, can be used by the controller in various ways, for example also for closing at least one opening device of the cleaning device, in particular with time control, for example after a predetermined number of seconds following identification of the pressurization. Thus, the controller, for example on hood-type washers, can trigger a time-controlled closing of the hood, for example after a certain number of seconds following coupling of a compressed gas line, in particular a compressed air line. The piston or the movable element configured in some other way can additionally be equipped in order to permit, for example, a visual identification of the pressure which is present, for example by means of color markings.

The interface, for example the Reed switch, can be integrated in particular in the cleaning chamber of the cleaning device, for example at or in a side wall. However, other positions are also conceivable, for example directly on the cleaning basket, but then signal transmission to the controller may comprise a certain challenge. However, even in this case, information can be transmitted to the controller via additional electrical contacts and/or a wireless transmission. By means of the proposed devices and the method, in particular cleaning operations of breathing regulators without pressurization, for example as a consequence of a compressed air tube not being coupled between cleaning device and cleaning basket, can therefore be reliably prevented.

The at least one further pressure sensor which can optionally be provided in the cleaning device is also simple to implement and can bring about considerable advantages. For example, at least one pressure sensor can be inserted into a machine-side compressed air line in the flow direction, for example downstream of a valve, in particular a magnetic valve, and, for example, upstream of a compressed air connection in the cleaning chamber to the cleaning basket. If a cleaning basket with a pressure connection is then inserted into the cleaning chamber and, for example, is automatically identified by the cleaning device, for example by means of an above-described basket coding, the controller can wait until the connection of the cleaning basket to the pressurization device is identified. For example, the controller can expect a short interruption to the pressure sensor signal, the interruption being able to arise, for example, if the cleaning basket with the pressure connection is connected to the pressurization device and the air initially propagates in the lines of the cleaning basket and is only compressed subsequently. This signal, which indicates a temporary drop in pressure, can be provided, for example, by the controller as a triggering signal for one or more actions. For example, this signal can be used as a triggering signal for a time-delayed closing of the opening device, for example the hood. Alternatively or additionally, an absence of the expected signal can trigger one or more actions by means of the controller. For example, in the event of an absence of the signal, the controller can prevent manual closing of the opening device, in particular the hood, and/or starting of the cleaning program, and/or can output an error message which indicates that the compressed air connection to the cleaning basket is absent and which, for example, only after renewed confirmation, can permit starting of the cleaning program.

The optional at least one pressure sensor in the cleaning device can therefore serve, for example, in general for monitoring the required gas pressure, for example the air pressure, in order in particular to identify any disturbance in the compressed gas supply, in particular the compressed air supply, and to take appropriate measures. For example, error messages can be indicated, and/or a further use of the cleaning device in the event of a malfunction can be permitted only after an additional confirmation.

Furthermore, in general, as explained above, the controller can also ensure record keeping. For example, there can be automatic documentation of rinsing parameters which may also include the correct pressurization of the at least one gas-conducting element, for example the correct coupling of the compressed air line, and/or the pressure of the pressurization. It can therefore in particular also be understood whether the process has been carried out correctly and whether, for example, correct working pressures have been maintained. Overall, the process reliability can therefore be significantly increased. In particular, pressure monitoring throughout the system of the pressurization, for example throughout the air system, can be realized. The monitoring can take place both in the cleaning basket and in the cleaning device, by means of simple pressure sensors and/or corresponding pressure switches.

In summary, the following embodiments are proposed without restricting further possible configurations:

Embodiment 1: A cleaning basket for cleaning a breathing apparatus, comprising at least one basket rack, furthermore comprising at least one pressure connection which is connectable to at least one gas-conducting element of the breathing apparatus, wherein the gas-conducting element can be charged with compressed gas at least while at least one cleaning fluid is being applied to the breathing apparatus, wherein the cleaning basket has at least one pressure sensor, wherein the pressure sensor is configured to identify a pressurization of the pressure connection, wherein the cleaning basket furthermore has at least one transmission device for transmitting at least one result of the identification of the pressurization to a cleaning device.

Embodiment 2: The cleaning basket according to the preceding embodiment, wherein the pressure sensor has at least one movable element, the movable element being movable by means of the pressurization.

Embodiment 3: The cleaning basket according to one of the preceding embodiments, wherein the movable element is movable by means of the pressurization counter to a restoring force.

Embodiment 4: The cleaning basket according to either of the two preceding embodiments, wherein the movable element has at least one element selected from the group consisting of a spring-actuated piston and a gravity-restoring piston.

Embodiment 5: The cleaning basket according to one of the preceding embodiments, wherein the movable element has at least one magnetic element.

Embodiment 6: The cleaning basket according to one of the preceding embodiments, furthermore comprising at least one indicator, wherein the indicator is configured to visually display a result of the identification of the pressurization of the pressure connection.

Embodiment 7: The cleaning basket according to one of the preceding embodiments, furthermore comprising at least one mask holder for holding at least one protective breathing mask in the cleaning basket.

Embodiment 8: The cleaning basket according to the preceding embodiment, furthermore comprising at least one suction element, wherein the suction element has at least one suction opening and is configured to extend into the protective breathing mask held by the mask holder, wherein the suction element is configured to be charged with negative pressure and to suck cleaning fluid out of at least one interior space of the protective breathing mask into the suction opening.

Embodiment 9: The cleaning basket according to one of the preceding embodiments, wherein the transmission device has at least one device selected from the group consisting of: a magnetic transmission element, in particular a movably mounted magnetic element which is changeable in a position by means of the pressurization; a mechanical actuating element, in particular a movably mounted mechanical element which is changeable in a position by means of the pressurization; an optical element, in particular a movably mounted and optically detectable element which is changeable in a position by means of the pressurization; an acoustic element; an electronic element; an electromagnetic element, in particular an electromagnetic element which is configured to generate and/or to change an electrical signal by means of the pressurization; an electronic element; a mechanical-electrical transducer, in particular an element which is configured to convert a pressurization into an electrical signal, in particular a mechanical-electrical transducer selected from the group consisting of an element which is based on the use of strain gauges and can change an electrical resistance in an electrical conductor by means of the pressurization, an element which can change an electrical resistance in an electrical semiconductor on the basis of crystalline structures by means of the pressurization, or an element which can generate an electrical voltage on the basis of crystalline structures by means of the pressurization; a mechanical-acoustic transducer, in particular an element which can generate an acoustic signal by means of the pressurization, for example directly and/or indirectly, in particular by means of an electrical-acoustic generation of sound.

Embodiment 10: A cleaning device for cleaning a breathing apparatus, comprising at least one cleaning chamber for receiving at least one cleaning basket according to one of the preceding embodiments, wherein the cleaning device furthermore has at least one application device for applying at least one cleaning fluid to the breathing apparatus, wherein the cleaning device furthermore has at least one interface for receiving the at least one result of the identification of the pressurization by the pressure sensor of the cleaning basket

Embodiment 11: The cleaning device according to the preceding embodiment, wherein the interface has at least one element selected from the group consisting of: a magnetic interface, in particular an interface with at least one magnetic sensor for detecting at least one magnetic field; a mechanical interface, in particular a mechanical interface with at least one mechanical sensor, in particular a mechanical sensor for detecting at least one position of a mechanical actuating element; an optical interface, in particular an optical interface with at least one optical sensor for optically detecting at least one position of at least one optically detectable element; an electrical interface, for example an electrically conductive connection; a radio interface, in particular an interface which is suitable for using the RFID technology; an acoustic interface, for example a microphone; an electronic interface.

Embodiment 12: The cleaning device according to one of the preceding embodiments relating to a cleaning device, wherein the interface has at least one Reed sensor, in particular a Reed sensor which is configured to detect at least one position of at least one movably mounted magnetic element of the cleaning basket, said magnetic element being changeable in a position by means of the pressurization.

Embodiment 13: The cleaning device according to one of the preceding embodiments relating to a cleaning device, furthermore comprising at least one controller, wherein the controller is configured to control at least one cleaning program in the cleaning device.

Embodiment 14: The cleaning device according to the preceding embodiment, wherein the controller is configured to receive the result of the identification of the pressurization by the pressure sensor of the cleaning basket from the interface.

Embodiment 15: The cleaning device according to the preceding embodiment, wherein the cleaning device furthermore has at least one pressurization device, wherein the pressurization device is connectable to the cleaning basket, wherein the pressurization device is configured to charge the at least one gas-conducting element of the breathing apparatus with compressed gas via the pressure connection of the cleaning basket while the at least one cleaning fluid is being applied to the breathing apparatus, wherein the cleaning device furthermore has at least one further pressure sensor, wherein the at least one further pressure sensor is configured to monitor the pressurization by means of the pressurization device.

Embodiment 16: The cleaning device according to the preceding embodiment, wherein the controller is configured to receive at least one signal from the further pressure sensor.

Embodiment 17: The cleaning device according to the preceding embodiment, wherein the controller is configured to identify a connection operation of a pressure connection of a cleaning basket to the pressurization device on the basis of a pressure fluctuation in the signal, in particular a sudden drop in pressure.

Embodiment 18: The cleaning device according to one of the four preceding embodiments, wherein the controller is configured so as, in accordance with the result of the identification of the pressurization and optionally furthermore in accordance with a signal from the further pressure sensor, to carry out at least one action selected from the group consisting of:
enabling the application of the at least one cleaning fluid to the breathing apparatus, in particular upon identification of a pressurization of the pressure connection, in particular upon identification of a pressurization of the pressure connection with a minimum pressure;
blocking the application of the at least one cleaning fluid to the breathing apparatus, in particular if a pressurization of the pressure connection is not identified, in particular if a pressurization of the pressure connection with a minimum pressure is not identified;
enabling at least one cleaning program of the cleaning device, in particular upon identification of a pressurization of the pressure connection, in particular upon an identification of a pressurization of the pressure connection with a minimum pressure;
blocking at least one cleaning program of the cleaning device, in particular if a pressurization of the pressure connection is not identified, in particular if a pressurization of the pressure connection with a minimum pressure is not identified;
closing at least one opening device of the cleaning device, in particular time-delayed closing, in particular closing upon identification of a pressurization of the pressure connection, in particular upon identification of a pressurization of the pressure connection with a minimum pressure, wherein an automatic starting of at least one cleaning program after the closing, in particular the time-delayed closing, is also possible;
outputting an item of information about the pressurization of the pressure connection to a user of the cleaning device, in particular outputting a warning indication, in particular if, when a pressurization of the pressure connection with a minimum pressure is not identified, an application of the at least one cleaning fluid to the breathing apparatus is intended to be started;
recording at least one item of information about the pressurization of the pressure connection, in particular recording a warning and/or if, after a warning, a cleaning program is started, in particular recording by means of the at least one controller;
blocking the cleaning device depending on at least one item of information about the pressurization of the pressure connection, in particular blocking a starting of a cleaning program if the minimum pressure is fallen short of;
automatically starting at least one cleaning program of the cleaning device, in particular upon identification of a pressurization of the pressure connection with a minimum pressure.

Embodiment 19: The cleaning device according to one of the five preceding embodiments, wherein the controller is configured to record cleaning operations in the cleaning device, wherein the recording comprises the respective identification of the pressurization by the pressure sensor of the cleaning basket and optionally also of the signal from the further pressure sensor or an item of information derived therefrom about the pressurization by means of the pressurization device.

Embodiment 20: The cleaning device according to the preceding embodiments relating to a cleaning device, furthermore comprising at least one basket identification device, wherein the basket identification device is configured to identify the at least one cleaning basket in the cleaning chamber.

Embodiment 21: The cleaning device according to the preceding embodiment, wherein the basket identification device is configured to differentiate between at least two different types of cleaning basket, in particular cleaning baskets with a pressure connection and cleaning baskets without a pressure connection.

Embodiment 22: A cleaning system for cleaning a breathing apparatus, comprising at least one cleaning basket according to one of the preceding embodiments relating to a cleaning basket, furthermore comprising at least one cleaning device according to one of the preceding embodiments relating to a cleaning device.

Embodiment 23: A method for cleaning a breathing apparatus, comprising the use of the cleaning system according to the preceding embodiment, furthermore comprising the following steps:
a) introducing the breathing apparatus into the cleaning basket;
b) connecting at least one gas-conducting element of the breathing apparatus to the pressure connection of the cleaning basket;
c) charging the gas-conducting element with compressed gas;
d) introducing the cleaning basket into the cleaning chamber of the cleaning device;
e) identifying the pressurization by means of the pressure sensor of the cleaning basket; and
f) transmitting the result of the pressurization identification to the cleaning device.

Embodiment 24: The method according to the preceding embodiment, furthermore comprising:
g) checking the result of the identification of the pressurization and, if the result reveals that the gas-conducting element has been charged with compressed gas, applying at least one cleaning fluid to the breathing apparatus.

Embodiment 25: The method according to one of the preceding embodiments relating to a method, furthermore comprising:
h) monitoring a pressurization of the pressure connection of the cleaning basket by a pressurization device of the cleaning device by means of at least one further pressure sensor of the cleaning device.

### BRIEF DESCRIPTION OF THE FIGURES

Further details and features emerge from the description below of exemplary embodiments, in particular in conjunction with the dependent claims. Here, the respective features can be realized by themselves alone or together in combination with one another. The invention is not restricted to the exemplary embodiments. The exemplary embodiments are schematically illustrated in the figures. The same reference numbers in the individual figures refer to elements which are identical or functionally the same or correspond to one another in respect of their functions.

Specifically:

FIG. 1 shows an embodiment of a cleaning system with embodiments of a cleaning device and a cleaning basket in a side view; and

FIG. 2 shows an embodiment of a cleaning basket in a perspective view.

FIGS. 3 and 4 show flow diagrams of methods for cleaning a breathing apparatus.

### DESCRIPTION OF THE EXEMPLARY EMBODIMENTS

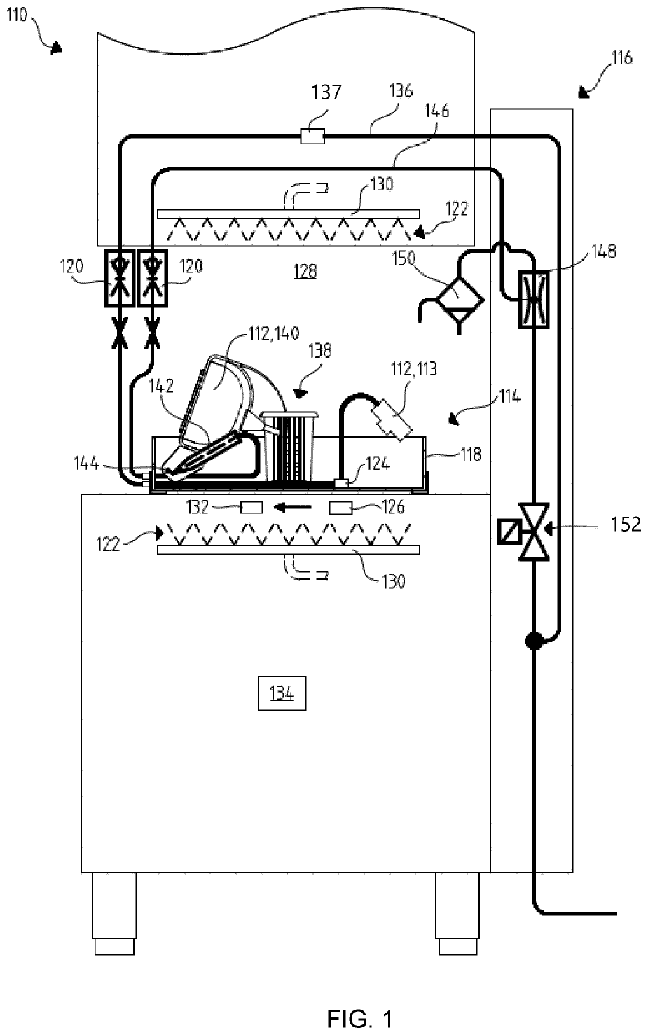

FIG. 1 shows an embodiment of a cleaning system 110 for cleaning a breathing apparatus 112. The cleaning system 110 comprises at least one cleaning basket 114 and at least one cleaning device 116. The cleaning basket 114 comprises at least one basket rack 118 and at least one pressure connection 120 which is connectable to at least one gas-conducting element 113 of the breathing apparatus 112. The gas-conducting element 113 can be charged here with compressed gas at least as at least one fluid 122 is being applied to the breathing apparatus 112. The cleaning basket 114 comprises at least one pressure sensor 124 which is configured to identify a pressurization of the pressure connection 120. Furthermore, the cleaning basket 114 comprises at least one transmission device 126 for transmitting at least one result of the identification of the pressurization to the cleaning device 116. The cleaning device 116 comprises at least one cleaning chamber 128 for receiving the at least one cleaning basket 114. Furthermore, the cleaning device 116 has at least one application device 130 for applying the at least one cleaning fluid 122 to the breathing apparatus 112. In addition, the cleaning device 116 comprises at least one interface 132 for receiving the at least one result of the identification of the pressurization by the pressure sensor 124 of the cleaning basket 114. A transmission of the at least one result of the identification of the pressurization is illustrated in FIG. 1 by way of example by means of an arrow from the transmission device 126 of the cleaning basket 114 to the interface 132.

Furthermore, the cleaning device 116 can comprise, for example, a controller 134. The controller 134 can be configured here in particular in order to control at least one cleaning program in the cleaning device 116. For example, the controller 134 can furthermore be configured to receive the result of the identification of the pressurization by the pressure sensor 124 of the cleaning basket 114 from the interface 132.

The cleaning device 116 can furthermore have, for example, at least one pressurization device 136. The pressurization device 136 can be connectable in particular to the cleaning basket 114. Furthermore, the pressurization device 136 can be configured to charge the at least one gas-conducting element 113 of the breathing apparatus 112 with compressed gas via the pressure connection 120 of the cleaning basket 114 while the at least one cleaning fluid 122 is being applied to the breathing apparatus 112. In particular, the cleaning device 116, for example for monitoring the pressurization by the pressurization device 136, can have at least one pressure sensor, for example a further pressure sensor 137. In particular, the controller 134 can be configured to receive at least one signal from the pressure sensor 137 of the cleaning device 116, in particular a signal from the pressure sensor 137 for monitoring the pressurization by means of the pressurization device 136. For example, the controller 134 can be configured to identify a connection operation of a pressure connection of a cleaning basket 114 to the pressurization device 136 on the basis of a pressure fluctuation in the signal, in particular a sudden drop in pressure.

As likewise illustrated in FIG. 1, the cleaning basket 114 can have, for example, at least one mask holder 138 for holding at least one protective breathing mask 140 in the cleaning basket 114. Furthermore, the cleaning basket 114 can comprise at least one suction element 142. In particular, the suction element 142 can have at least one suction opening 144 and can be configured to extend into the protective breathing mask 140 held by the mask holder 138. For example, the suction element 142 can furthermore be configured to be charged with negative pressure and to suck cleaning fluid 122 out of at least one interior space of the protective breathing mask 140 into the suction opening 144. Cleaning fluid 122, which accumulates in an interior space of the protective breathing mask 140, for example, while being applied to the breathing apparatus 112, for example to the gas-conducting element 113 and the protective breathing mask 140, can thus preferably be sucked out of the interior space of the protective breathing mask 140. For example, for this purpose, the cleaning device 116 can furthermore comprise at least one negative pressure line 146, for example a vacuum line, which can be connectable to the suction element 142 via a further pressure connection 120. Furthermore, the cleaning device 116, can have a Venturi nozzle 148, for example for generating the negative pressure. A water separator 150 which is preferably likewise included by the cleaning device 116 can be configured, for example, in order to return the cleaning fluid 122 sucked out of the interior space of the protective breathing mask 140 into the cleaning chamber 128. Furthermore, the cleaning device 116 can have a valve 152, for example a magnetic valve, for selectively opening and/or closing the negative pressure line.

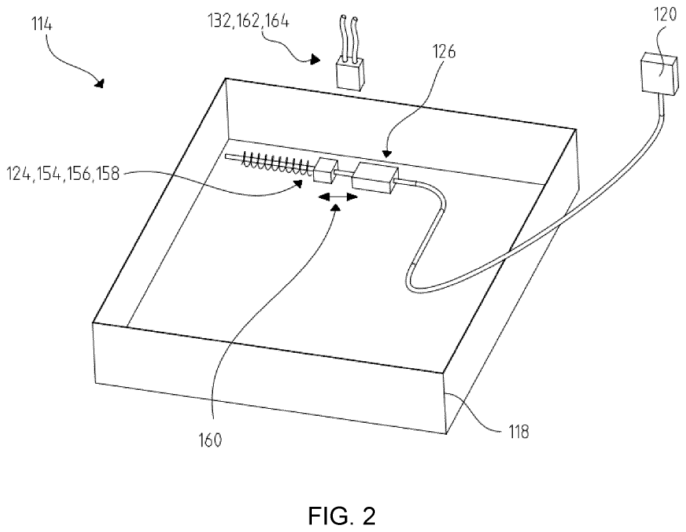

FIG. 2 shows an exemplary embodiment of a cleaning basket 114. In particular, the pressure sensor 124 of the cleaning basket 114 can have, for example, a movable element 154, wherein the movable element 154 can be movable, for example, by means of the pressurization. For example, the movable element 154 can be movable by means of the pressurization counter to a restoring force. In particular, the movable element 154 can be, for example, a spring-actuated piston 156, and therefore the restoring force can correspond, for example, to a force of a spring force. The movable element 154 can have, for example, at least one magnetic element 158. In the exemplary embodiment of the cleaning basket 114 that is shown in FIG. 2, a possible extent of a movement, in particular a movement path 160, of the magnetic element 158 is illustrated, by way of example, by an arrow. As already explained above, the cleaning basket 114 comprises at least one transmission device 126 for transmitting at least one result of the identification of the pressurization to the cleaning device 116. For example, the transmission device 126 can have a magnetic transmission element, wherein the transmission device can be configured as a counterpart to the interface 132 included by the cleaning device 116. In particular, the interface 132 can comprise, for example, a magnetic switching element 162, in particular a Reed sensor 164. Thus, in the embodiment illustrated here of the cleaning basket 114, for example the above-described movement of the movable element 154 with the at least one magnetic element 158 is detectable by the Reed sensor 164.

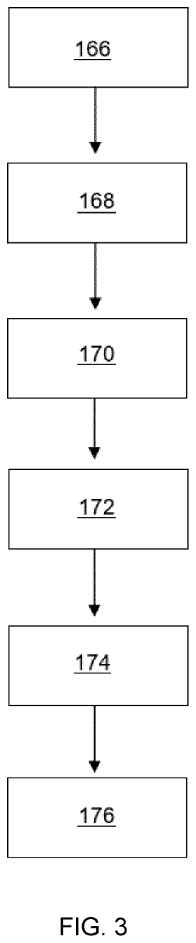

FIG. 3 shows a flow diagram of a method for cleaning a breathing apparatus 112, comprising the use of the cleaning system 110. The method comprises at least the following steps:
a) (indicated by reference sign 166) introducing the breathing apparatus 112 into the cleaning basket 114;
b) (indicated by reference sign 168) connecting at least one gas-conducting element 113 of the breathing apparatus 112 to the pressure connection 120 of the cleaning basket 114;
c) (indicated by reference sign 170) charging the gas-conducting element 113 with compressed gas;
d) (indicated by reference sign 172) introducing the cleaning basket 114 into the cleaning chamber 128 of the cleaning device 116;
e) (indicated by reference sign 174) identifying the pressurization by means of the pressure sensor 124 of the cleaning basket 114; and
f) (indicated by reference sign 176) transmitting the result of the identification of the pressurization to the cleaning device 116.

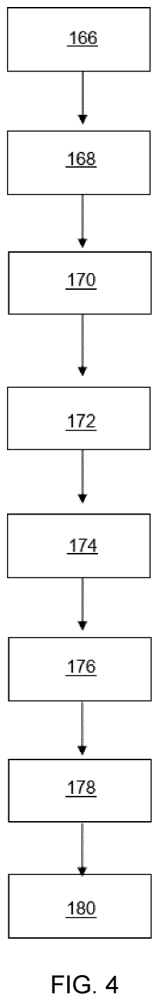

FIG. 4 shows another flow diagram of a method for cleaning a breathing apparatus 112, in which method the cleaning system 110 is used. In particular, the method can furthermore comprise one or more of the following steps:
g) (indicated by reference sign 178) checking the result of the identification of the pressurization and, if the result reveals that the gas-conducting element 113 has been charged with compressed gas, applying at least one cleaning fluid 122 to the breathing apparatus 112; and
h) (indicated by reference sign 180) monitoring a pressurization of the pressure connection 120 of the cleaning basket 114 by a pressurization device 136 of the cleaning device 116 by means of at least one further pressure sensor 137 of the cleaning device 116.

### LIST OF REFERENCE SIGNS

110 Cleaning system
112 Breathing apparatus
113 Gas-conducting element
114 Cleaning basket
116 Cleaning device
118 Basket rack
120 Pressure connection
122 Cleaning fluid
124 Pressure sensor
126 Transmission device
128 Cleaning chamber
130 Application device
132 Interface
134 Controller
136 Pressurization device
137 Pressure sensor
138 Mask holder
140 Protective breathing mask
142 Suction element
144 Suction opening
146 Negative pressure line
148 Venturi nozzle
150 Water separator
152 Valve
154 Movable element
156 Spring-actuated piston
158 Magnetic element
160 Movement path
162 Magnetic switching element
164 Reed sensor
166 Step a)
168 Step b)
170 Step c)
172 Step d)
174 Step e)
176 Step f)
178 Step g)
180 Step h)

1. A cleaning basket for cleaning a breathing apparatus, comprising at least one basket rack, furthermore comprising at least one pressure connection which is connectable to at least one gas-conducting element of the breathing apparatus, wherein the gas-conducting element can be charged with compressed gas at least while at least one cleaning fluid is being applied to the breathing apparatus, wherein the cleaning basket has at least one pressure wherein the pressure sensor, is configured to identify a pressurization of the pressure connection, wherein the cleaning basket furthermore has at least one transmission device for transmitting at least one result of the identification of the pressurization to a cleaning device.

2. The cleaning basket as claimed in claim 1, wherein the pressure sensor has at least one movable element, the movable element being movable by means of the pressurization.

3. The cleaning basket as claimed claim 1, wherein the movable element is movable by means of the pressurization counter to a restoring force.

4. The cleaning basket as claimed claim 1, wherein the movable element has at least one magnetic element.

5. The cleaning basket as claimed in claim 1, furthermore comprising at least one mask holder for holding at least one protective breathing mask in the cleaning basket, furthermore comprising at least one suction element wherein the suction element has at least one suction opening and is configured to extend into the protective breathing mask held by the mask holder wherein the suction element is configured to be charged with negative pressure and to suck cleaning fluid out of at least one interior space of the protective breathing mask, into the suction opening.

6. The cleaning basket as claimed in claim 1, wherein the transmission device has at least one device selected from the group consisting of: a magnetic transmission element; a mechanical actuating element; an optical element; an acoustic element; an electronic element; an electromagnetic element; a mechanical-electrical transducer; a mechanical-acoustic transducer.

7. A cleaning device for cleaning a breathing apparatus, comprising at least one cleaning chamber for receiving at least one cleaning basket as claimed claim 1, wherein the cleaning device furthermore has at least one application device for applying at least one cleaning fluid to the breathing apparatus, wherein the cleaning device furthermore has at least one interface for receiving the at least one result of the identification of the pressurization by the pressure sensor of the cleaning basket.

8. The cleaning device as claimed in claim 7, wherein the interface has at least one element selected from the group consisting of: a magnetic interface; a mechanical interface; an optical interface; an electrical interface; a radio interface; an acoustic interface; an electronic interface.

9. The cleaning device as claimed in claim 7, wherein the interface has at least one Reed sensor which is configured to detect at least one position of at least one magnetic element of the cleaning basket said magnetic element being mounted movably and being changeable in a position by means of the pressurization.

10. The cleaning device claimed claim 7, furthermore comprising at least one controller wherein the controller is configured to control at least one cleaning program in the cleaning device, wherein the controller is configured to receive the result of the identification of the pressurization by means of the pressure sensor of the cleaning basket from the interface.

11. The cleaning device as claimed claim 10, wherein the cleaning device furthermore has at least one pressurization device, wherein the pressurization device is connectable to the cleaning basket, wherein the pressurization device is configured to charge the at least one gas-conducting element of the breathing apparatus with compressed gas via the pressure connection of the cleaning basket while the at least one cleaning fluid is being applied to the breathing apparatus wherein the cleaning device furthermore has at least one further pressure sensor, wherein the at least one further pressure sensor, is configured to monitor the pressurization by means of the pressurization device.

12. The cleaning device as claimed in claim 11, wherein the controller is configured to receive at least one signal of the further pressure sensor, wherein the controller is configured to identify a connection operation of a pressure connection of a cleaning basket to the pressurization device on the basis of a pressure fluctuation in the signal.

13. The cleaning device as claimed claim 10, wherein the controller is configured so as, in accordance with the result of the identification of the pressurization, to carry out at least one action selected from the group consisting of:
enabling the application of the at least one cleaning fluid to the breathing apparatus;
blocking the application of the at least one cleaning fluid to the breathing apparatus;
enabling at least one cleaning program of the cleaning device blocking at least one cleaning program of the cleaning device;
closing at least one opening device of the cleaning device;
outputting an item of information about the pressurization of the pressure connection to a user of the cleaning device;
recording at least one item of information about the pressurization of the pressure connection;
blocking the cleaning device depending on at least one item of information about the pressurization of the pressure connection;
automatically starting at least one cleaning program of the cleaning device.

14. A cleaning system cleaning a breathing apparatus, comprising at least one cleaning basket as claimed in claim 1, furthermore comprising at least one cleaning device as claimed in claim 7.

15. A method for cleaning a breathing apparatus, comprising the use of the cleaning system as claimed in claim 14, furthermore comprising the following steps:
a) introducing the breathing apparatus into the cleaning basket;
b) connecting at least one gas-conducting element of the breathing apparatus to the pressure connection of the cleaning basket;
c) charging the gas-conducting element with compressed gas;
d) introducing the cleaning basket into the cleaning chamber of the cleaning device;
e) identifying the pressurization by means of the pressure sensor of the cleaning basket; and
f) transmitting the result of the pressurization identification to the cleaning device.
